(19) United States
(12) Patent Application Publication
(10) Pub. No.: US 2014/0066729 A1
(43) Pub. Date: Mar. 6, 2014

(54) Sampling Device And System For Capturing Biological Targets Of A Body Fluid, And Process For Manufacturing This Device

(71) Applicant: Commissariat A L'Energie Atomique Et Aux Energies Alternatives, Paris (FR)

(72) Inventors: Marie-Line Cosnier, Grenoble (FR); Dominique Lauro, Le Moutaret (FR)

(21) Appl. No.: 14/014,547

(22) Filed: Aug. 30, 2013

(30) Foreign Application Priority Data
Aug. 30, 2012 (FR) 12 58114

(51) Int. Cl. A61B 5/157; A61B 5/15

(52) U.S. Cl. CPC A61B 5/157; A61B 5/150274
USPC 600/309; 264/261

(57) ABSTRACT

The invention relates to a sampling device adapted to be inserted into a hollow tubular endpiece of the needle or catheter type, and to emerge from the endpiece with a view to contact with a bodily fluid containing biological samples to be sampled, to a sampling system incorporating this endpiece and this device, which is mounted so as to slide in the latter, and to a method for manufacturing this device.

This sampling device comprises a framework (5) microstructured by openings (5b), and a biocompatible and porous crosslinked polymer layer which comprises capture supports adapted to capture the said targets and which is adapted to retain these supports from the fluid and to let through only fluid particles including these targets with a size of less than a cutoff size, the said polymer layer filling all or some of the said openings, so as to be retained by the said framework.

According to the invention, the framework is substantially undeformable between positions in which it is inserted into the endpiece and in which it emerges from the latter.

The present invention relates to a sampling device adapted to be inserted into a hollow tubular endpiece of the needle or catheter type, and to emerge from the endpiece with a view to contact with a bodily fluid containing biological targets to be sampled, to a sampling system incorporating this endpiece and this device, which is slidably mounted in the latter, and to a method for manufacturing this device. The invention applies to sampling carried out, particularly in vivo, in bodily fluids of the human body, for example circulating bodily fluids, in particular the blood, the cerebrospinal fluid, the interstitial fluid or the lymph, in which case these fluids may, as targets, contain proteins, oligonucleotides such as RNA or DNA, antibodies, enzymes or cells, without limitation.

For a number of years, analysis techniques, whether based on genomics, proteomics or immunology, have been progressing and have reached remarkable levels of sensitivity. These techniques are based on the recognition of biological elements of interest, or targets, which need to be extracted from the other elements present in the sample taken, whether this is done in vivo or ex vivo. If the target is absent or in too low a proportion in relation to the sensitivity of the analysis method, then the measurement will not be possible.

The sample preparation methods aim to capture the intended targets and bring them in contact after concentration with a functionalized surface, on which a measurement is carried out. They are highly advantageous because, by concentration of this target, they make it possible to relax the constraint on the measurement sensitivity. On the other hand, they are ineffective when the target is not present in the sample taken. This drawback is evident, for example, in the case of blood analysis which has seen a tendency to reduce the sampled volume, raising difficulties due to the presence or absence of the intended element and the sensitivity of the measurement system.

One conventional method is to mix nanoparticles such as nanobeads, carrying recognition sites, with the sample containing the target, then to carry out the recognition in volume and finally to recover these nanoparticles by centrifuging or magnetic attraction before carrying out controlled rerelease of the capture targets onto a measurement surface.

Another known method consists in recirculating the liquid to be tested over a surface comprising the recognition sites in question.

The problem of testing bodily fluids flowing in the human body may be addressed in the same way. In the example in which the volume to be tested is whole blood or cerebrospinal fluid, the same approach may similarly be envisaged, consisting in injecting metallic and/or magnetic nanoparticles into the bodily fluid or under the skin, allowing them to recognize the targets then recovering them, for example by applying a local magnetic field or by filtration on an extracorporeal circuit. This approach requires very in-depth study of the particles injected, in terms of toxicity and filtration in the kidneys, the liver, etc. One problem which has not yet been resolved is satisfactory recovery of such injected particles.

In order to overcome problems, particularly of the risk of triggering immune reactions and toxicity which may result from the injection of such nanoparticles into the human body, the solutions developed to date generally rely on encapsulation of these metallic and/or magnetic nanoparticles in various materials, particularly in biocompatible polymers. Depending on the porosity of this polymer, it may act as a filter blocking biological species exceeding a certain size. Here, a biological species is intended to mean cells, molecules, viruses, bacteria or antibodies.

In the known devices, the use of a coating or encapsulation improves the performance of the measurement device, whether for the contrast efficiency, tolerance to tissues or capture capacity. However, a major drawback of these devices is that they are not designed to recover the nanoparticles after they have come in contact with the intended targets in the fluid in question, in particular in order to capture these targets or following an injection prior to an MRI (magnetic resonance imaging) analysis. In other words, the problem of what finally happens to the nanoparticles injected in vivo arises in these known devices.

Document WO-A1-2011/086486 in the name of the Applicant makes it possible to overcome this drawback by presenting a device for bringing supports for capturing targets to be analyzed temporarily in contact with a bodily fluid containing them, this device comprising a sampling endpiece, an end of which for contact with the fluid is bound to the capture supports, which are covered with a biocompatible and porous crosslinked polymer layer. This layer is designed to retain the capture supports and to let through only particles including these targets with a size of less than a cutoff size, so that the capture supports can be recovered by dissolving this layer after the contact. This document mentions possible integration of the capture supports in a meshed structure connected to the endpiece, the strands of which are covered with the said layer that encapsulates these capture supports, which may consist of functionalized nanoparticles. This meshed structure is folded into an e.g. tubular guide structure and deployed reversibly from the latter during the sampling. The meshed structure is intended to be deployed in the body, for example in a blood vessel, in the same way as a stent. It is therefore clear that the biological fluid flows between the strands, that is to say between the meshes of the device.

Although the devices presented in this document provide entirely satisfactory results, the Applicant has sought to optimize the attachment or adhesion of the polymer layer as a whole to such a meshed structure, so as to minimize the risks of detachment of polymer fragments from the surface which this layer covers.

Document WO-A1-2010/145824 presents a detection device for the enrichment of samples, comprising a three-dimensional detection surface which is rendered functional by a multitude of detection receptors which it comprises, and which may be microstructured, for example in the manner of a meshed network. This document does not relate to the adhesion to a framework of a polymer layer coating capture supports which are separate from this framework.

It is an object of the present invention to provide a sampling device adapted to be inserted into a hollow tubular endpiece of the needle or catheter type, and to emerge from the endpiece with a view to contact, particularly in vivo, with a bodily fluid containing biological targets to be sampled, which overcomes the aforementioned drawbacks, the device comprising a framework microstructured by openings, and a biocompatible and porous crosslinked polymer layer which comprises capture supports adapted to capture the said targets and which is adapted to retain these supports from the fluid and to let through only fluid particles including these targets with a size of less than a cutoff size.

To this end, a device according to the invention is such that the said polymer layer fills all or some of the said openings, so as to be retained by the said framework.

According to one embodiment of the invention, the framework is circumscribed by at least one cylindrical surface having a largest transverse dimension of between 500 μm and 2 mm, the framework being embedded in the said polymer layer.

In other words, the polymer layer fills all or some of the openings of the framework while extending on either side of the latter.

The framework may have an external face and delimit an internal volume, the polymer layer extending through the said openings from this internal volume to this external face and beyond the latter.

The framework may be tubular, in which case it encloses a cylindrical internal volume, the said polymer layer then extending on either side of the framework. It will be noted that this overall tubular framework microstructured by openings (i.e. by micro-openings extending fully through with dimensions of the order of one or several tens of μm) lets the uncrosslinked (i.e. not yet gelled) polymer material of the porous layer pass through so as to partly or fully fill the internal space of this framework, which makes it possible to significantly improve the attachment of this layer to the framework by this optimized adhesion on the two faces, radially internal and external, of the framework. The result of this is to minimize the risks of detachment of the layer from the framework, and therefore of release of a fragment of this layer into the bodily fluid from which the sampling is being carried out.

In the present description, an “endpiece” is intended to mean an endpiece which may correspond to all or part of an injection or sampling needle, of a catheter or of an external system, which is adapted to be introduced into the medium containing the bodily fluid. Such a medium may, for example, be the vein of a human being or animal.

In the present description, “at least one cylindrical surface” is intended to mean one or more cylindrical surfaces (i.e. each being defined over its height by a generatrix and over its cross section by a directrix in the form of any curved line, for example a directrix in the form of an ellipse or circle), given that the cross section of the external face of the framework may be constant (the case in which this face is circumscribed by a single cylindrical surface) or variable (the case in which this face is successively circumscribed by a plurality of cylindrical surfaces and/or by one or more frustoconical surfaces following one or more cylindrical surfaces).

The crosslinked layer of the device according to the invention has a selective permeability and makes it possible to avoid direct contact between the capture supports and the fluid, in order to prevent an immune reaction, to prevent dissemination of all or some of the capture supports into the fluid, to selectively capture the targets according to their size and to impose less mechanical stresses on the surrounding biological tissues owing to its flexibility.

According to another characteristic of the invention, the said framework has a single longitudinal symmetry axis which is intended to be parallel to that of the said endpiece, the said framework maintaining an overall tubular geometry in its positions in which it is inserted into the endpiece and in which it emerges from the latter.

In other words, and in contrast to the meshed structure of the aforementioned document WO-A1-2011/086486, which is deployed from the endpiece and is folded into the latter, a framework according to the invention is incapable of being deployed from the endpiece (i.e. extended or opened, for example unwound) and folded into the latter (i.e. retracted more compactly, for example wound) because it is substantially undeformable and has a geometry of revolution matching the internal face of the corresponding end zone of the endpiece.

According to another characteristic of the invention, the crosslinked polymer layer has a viscosity, measured by a cone and plate rheometer, which is equal to or greater than 100 mPa·s and preferably lies between 150 mPa·s and 5800 mPa·s. Beyond these values, the viscosity is too high, which limits the possibilities of shaping the polymer.

It will be noted that this particular viscosity of the polymer layer contributes significantly to good attachment of this layer to the microstructured framework of the device according to the invention, which is characterized by the surface irregularities and protrusions formed by the radially external and internal edges of the micro-openings.

Advantageously, this micro-openworked framework of a device according to the invention may be of the latticed, woven or plaited fabric type, comprising a multitude of openings or interstices, separated in pairs by a pitch of between 30 μm and 60 μm.

According to a particularly advantageous exemplary embodiment of the invention, the said framework is of the woven or latticed metallic fabric type. It may then be substantially planar, or form a closed surface so as to form an internal volume. It may it this case comprise one or more micro-latticed cylindrical tubes of substantially circular cross section, with, in the case of a plurality of tubes, their respectively longitudinal axes of symmetry being parallel. This may involve concentric tubes.

Preferably, the said framework has a thickness of between 10 μm and 100 μm, the said at least one cylindrical surface having a substantially elliptical or circular cross section.

It will, however, be noted that a framework according to the invention may furthermore, on its external and/or internal faces, have zones which are not micro-openworked but are microstructured in another way, for example by indentations (i.e. holes not extending radially through) and/or continuous or discontinuous reliefs with dimensions—such as the radial depth—of less than 1 mm, these indentations and/or reliefs preferably having such dimensions lying between 20 μm and 90 μm on these external and/or internal faces.

In particular, such a framework microstructured by these indentations and/or reliefs in addition to the said micro-openings may be obtained by sandblasting one or more initially smooth zones of its external face.

According to another characteristic of the invention, the said polymer layer may form, with respect to the said external face of the framework, an external coating substantially coaxial with this framework and having a thickness of between 50 μm and 300 μm.

According to the invention, the said framework may be embedded in the said polymer layer over a part of its axial length lying between 1 mm and 5 cm, it being possible for this polymer layer to have a volume of between 1 ml and 10 ml.

It will be noted that such a volume makes it possible to contain a large number of capture supports, such as nanobeads, which is particularly useful when desiring to capture minority species flowing in a bodily liquid.

According to another characteristic of the invention, the said capture supports may comprise magnetic or non-magnetic nanoparticles which are functionalized on the surface by grafted functions adapted to capture the said targets, and which have a largest transverse dimension of between 50 nm and 500 nm and are embedded in the mass of the said polymer layer, these nanoparticles preferably being nanobeads or nanospheres based on an iron oxide with a diameter of between 80 nm and 200 nm and functionalized by anionic or cationic functions (or as a variant by antibodies, oligonucleotides such as aptamers, surface functions of the chromatographic type and functions from the peptide and oligonucleotide libraries).

Advantageously, the framework may be made of metallic material, preferably stainless steel of surgical grade, silicon or a polymer material such as a silicone, and the polymer layer is based on at least one biocompatible polymer with reversible gelling, selected from the group consisting of alginate gels, copolymers of alginate and poly-L-lysine, chitosan, agarose, cellulose, poly(trimethylammonium ethylacrylate methyl sulfate)-b-poly(acrylamide), poly(hydroxyethylmethacrylate (HEMA), poly(hydroxyethylmethacrylate-methyl methacrylate (HEMA-MMA) and other copolymers based on methacrylate, polyethylene glycols, copolymers of acrylonitrile and polyethylene glycol, polysaccharides and mixtures thereof.

Even more advantageously, the framework may be provided on its surface with functional groups creating chemical bonds between the framework and the polymer layer, preferably carboxylic acid or amine groups in the case in which the framework is metallic, for bonding with hydroxyl groups of this layer.

Preferably, the polymer layer is based on at least one alginate gel which is obtained by means of polycations, preferably selected from the group consisting of polycations of calcium, barium, iron and strontium. This is because the use of an alginate is particularly advantageous since it is perfectly biocompatible, non-toxic and lets the targets to be captured pass through it. Furthermore, it can be polymerized and gelled at ambient temperatures and remains in gelled form at body temperatures and at the pH corresponding to physiological conditions.

Also advantageously, the crosslinked polymer layer may have a Young's modulus, measured on the basis of compression tests carried out with a rheometer, of between 50 kPa and 270 kPa inclusive. Beyond these values, the polymer becomes difficult to shape.

Advantageously, the polymer layer may have a porosity, defining the said cutoff size, which lies between 10 nm and 1 μm, with a surface porosity of between 10 nm and 50 nm and a porosity in the bulk of between 100 nm and 1 μm, in the preferred example of an alginate gel.

A sampling system according to the invention comprises:
a hollow tubular endpiece of the needle or catheter type, which has an internal diameter of between 500 μm and 2 mm,
a sampling device inserted into the endpiece and capable of emerging by sliding from an end of this endpiece with a view to contact, particularly in vivo, with a bodily fluid containing biological targets to be sampled, and
a thrust member capable of making the said sampling device slide reversibly out of the said endpiece.

This system of the invention is characterized in that this device is as defined above and is optionally provided with a means for connection to the said end of the endpiece.

According to another characteristic of the invention, the said thrust member may be of the syringe type, and comprises:
a pump body, in which the said endpiece is mounted, and
a rod which can be inserted into the said endpiece in order to make the said sampling device slide therein in a reversible translation parallel to the symmetry axis of the endpiece.

Preferably, a liquid of the physiological liquid type is arranged between the said insertable rod and the said sampling device. This makes it possible to limit the degradation of the polymer during the thrust. The liquid is advantageously injectable.

A method according to the invention for manufacturing a sampling device such as the one defined above comprises the following steps:

a) preparation of an uncrosslinked polymer composite incorporating the said capture supports and the said polymer layer covering them in the uncrosslinked state,

b) insertion of the said framework, without this composite, into a tubular mold, optionally with connection of the framework to a sampling end of the endpiece (preferably, the mold is perforated so that the gelling solution is in contact with the periphery of the polymer during the gelling of the latter, this making it possible to control the surface porosity better; the size of the perforations is advantageously less than 1 mm),

c) assembly of the endpiece containing this framework in a sampling member of the syringe type,

d) take-up of the uncrosslinked composite prepared in a) by this sampling member, in order to inject this composite inside the endpiece in contact with the framework, then

e) crosslinking in a gelling bath of the endpiece which is filled with the uncrosslinked composite injected in d) and which has previously been extracted from this sampling member, in order to obtain the said crosslinked polymer layer fixed to the framework.

Advantageously, step a) may comprise:

a1) dispersion in an aqueous buffer solution of the said capture supports comprising magnetic or non-magnetic functionalized nanoparticles, then

a2) addition under agitation to the dispersion obtained in a1) of at least one biocompatible polymer with reversible gelling, in order to obtain the uncrosslinked composite in which these nanoparticles are embedded.

Other advantages, characteristics and details of the invention will emerge from the remainder of the description which follows with reference to appended drawings, which are given solely by way of examples and in which:

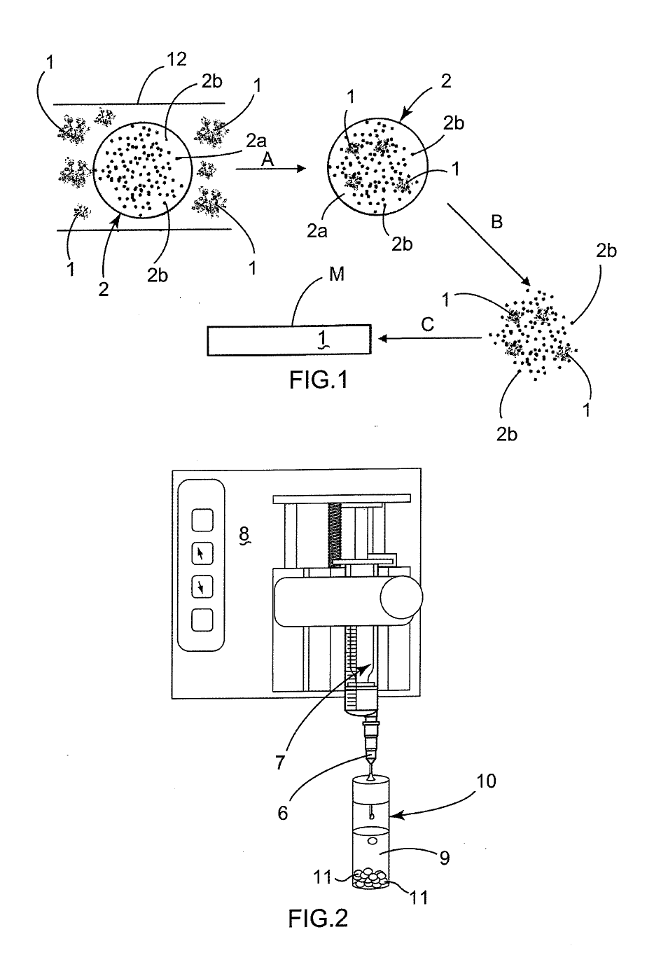

FIG. 1 is a schematic view illustrating various phases of a method for sampling biological targets in a bodily fluid according to an exemplary embodiment of the invention,

FIG. 2 is a partial front view of an installation for the preparation of a crosslinked polymer composite included in a sampling device according to the invention,

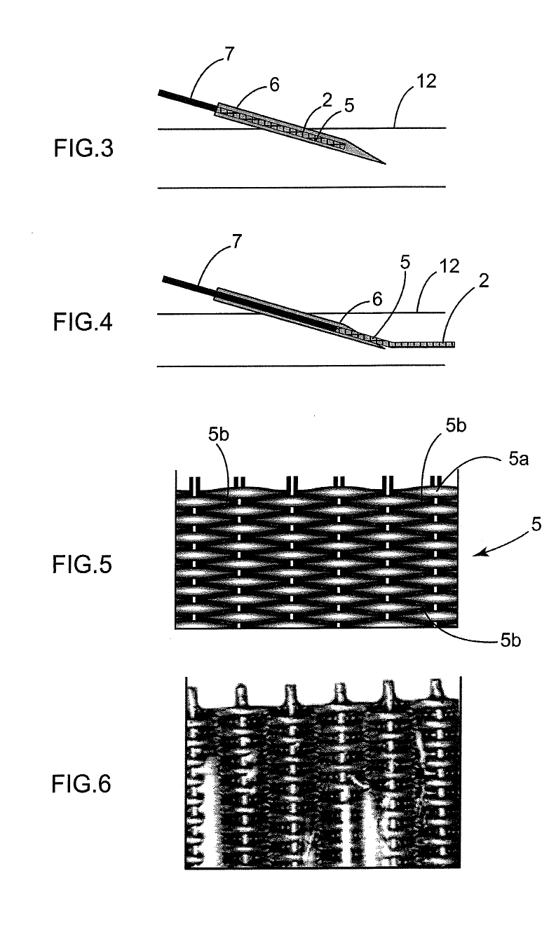

FIG. 3 is a schematic view in longitudinal section of an endpiece of the needle type, which contains a sampling device according to the invention and which has been introduced into a vein of the human body, the device being in the position inside the endpiece,

FIG. 4 is a schematic view in longitudinal section of the endpiece of FIG. 3, still introduced into this vein, but with the sampling device being in the position partially outside the endpiece,

FIG. 5 is a detail view of a micro-openworked framework of a sampling device according to an example of the invention, this framework being without the crosslinked polymer composite intended to cover it,

FIG. 6 is a photograph showing the coverage of the framework of FIG. 5 by this crosslinked composite,

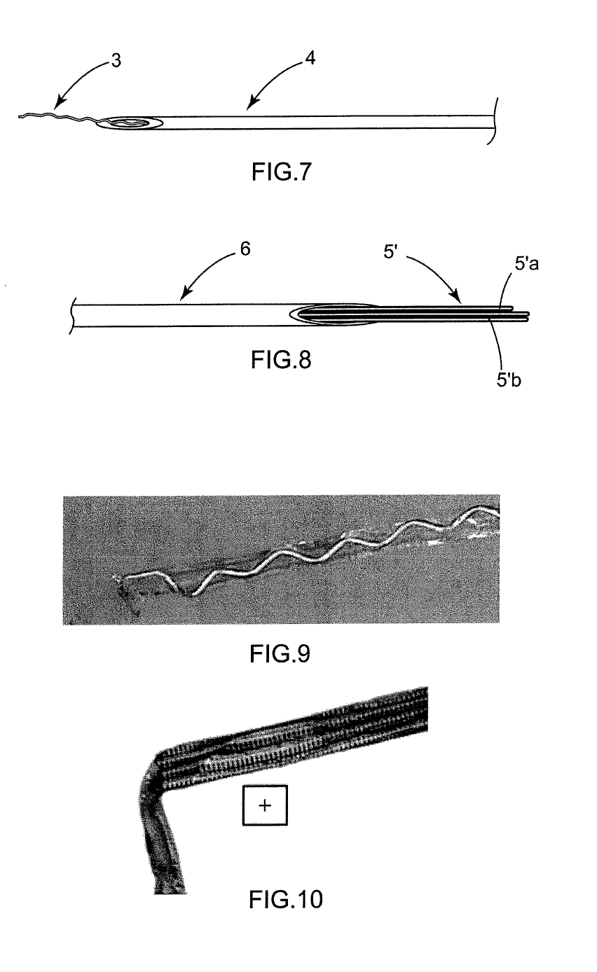

FIG. 7 is a partial plan view of an endpiece of the needle type containing a wire framework not according to the invention, without this crosslinked composite,

FIG. 8 is a partial plan view of an endpiece of the needle type containing a micro-openworked framework according to another example of the invention, without this crosslinked composite,

FIG. 9 is a photograph showing the coverage of the wire framework of FIG. 7 by this crosslinked composite, immediately after injection and crosslinking of the composite in the endpiece in contact with the framework,

FIG. 10 is a photograph showing the coverage of the micro-openworked framework of FIG. 8 by this crosslinked composite, immediately after injection and crosslinking of the composite in the endpiece in contact with the framework,

FIG. 11 is a photograph showing the coverage of the wire framework of FIG. 7 by this crosslinked composite, after washing of the sampling device shown in FIG. 9 with water, and

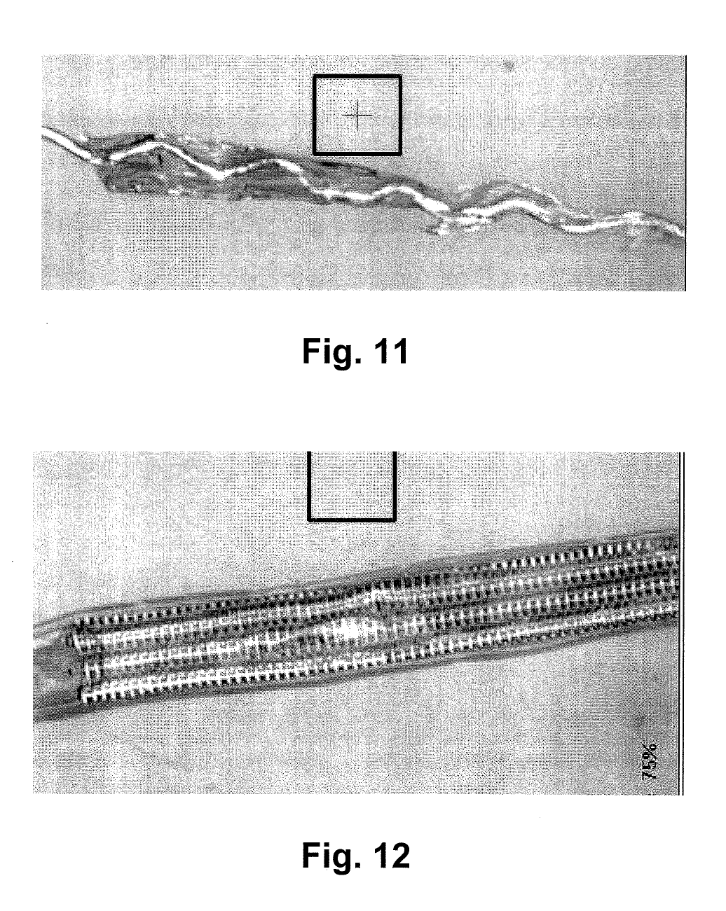

FIG. 12 is a photograph showing the coverage of the micro-openworked framework of FIG. 8 by this crosslinked composite, after washing of the sampling device shown in FIG. 10 with water.

In the sampling method illustrated in FIG. 1, in a first step A, a crosslinked (i.e. gelled) polymer composite 2 is incubated in a fluid containing targets 1 (e.g. proteins), the composite consisting in this example of a layer 2a of a calcium alginate gel in which capture supports 2b formed by magnetic nanoparticles, advantageously based on Fe₂O₃, are embedded (this composite 2 is fixed to a micro-openworked framework according to the invention, not represented here and described below with reference to FIGS. 5, 6, 8, 10 and 12). After this incubation, the filter effect of the alginate gel leads to penetration of the targets 1 into the layer 2a until they are captured by the nanoparticles 2b. The latter have an average diameter of about 100 nm and can be functionalized or grafted with surface functions, for example of the polystyrene sulfonate type.

Next, operations B of washing and degelling the alginate layer 2a were carried out, which led to the nanoparticles 2b bound to the targets 1 in the alginate solution being obtained, the binding advantageously being by means of a polycation chelating agent which is for example, for sodium polycations, ethylene diamine tetraacetic acid (EDTA) or sodium citrate.

Lastly, separation C was carried out, advantageously by magnetization (the magnet M used is symbolized by a rectangle in FIG. 1), of the nanoparticles 2b bound to the targets 1, which makes it possible to obtain, with a view to subsequent analyses, these targets 1 such as proteins adsorbed on the capture surface of the nanoparticles 2b.

Polymer composites 2 incorporating the nanoparticles 2b embedded in the porous polymer layer 2a were prepared in the following way. First, these nanoparticles 2b were added and dispersed by agitation in an aqueous buffer composed of 154 mM of NaCl and HEPES. Next, a powdered alginate was added to the dispersion obtained in this way, with a mass fraction varying from 1% to 3%, and in particular equal to 1.5% for the two examples of the invention relating to FIGS. 6 and 10, with rotary and ultrasonic agitation for at least 10 hours. The composites 2 in the uncrosslinked state, consisting of a polymerized alginate hydrogel 2a coating the nanoparticles 2b, were obtained in this way.

Then, independently of the ungelled composites 2 prepared in this way, the following were inserted:
in the example not according to the invention of FIG. 7, a framework 3 consisting of a single metal wire with a diameter of 140 μm (made of surgical stainless steel AISI 316L), into a needle 4 having an external diameter equal to 0.8 mm,
in the example according to the invention of FIG. 5, an overall tubular framework 5 (only the radially external face 5a of which is shown) formed by a woven or plaited fabric (made of surgical stainless steel AISI 316L), having interstices 5b at a pitch of about 50 μm, through which the composite 2 will penetrate in order to furthermore coat the radially internal face of the framework 5, into a needle 6 having an external diameter equal to 1.1 mm, and
in the other example according to the invention of FIGS. 8, 10, 12, a micro-latticed framework 5′ (also made of surgical stainless steel AISI 316L), into an identical needle 6, (the photographs of FIGS. 10 and 12 make it possible to distinguish the micro-latticed structure of this framework 5′, which extends over a surface 5′a and has openings 5′b as can be seen in FIG. 8).

In these three exemplary cases, the frameworks 3, 5, 5′ are circumscribed by a cylindrical volume having a largest transverse dimension of between 500 μm and 5 mm.

Each of these needles 4, 6 was assembled in a 5 ml syringe 7 (see FIG. 2) provided with a syringe plunger 8, and the composite 2 to be gelled was injected via this syringe 7 into the needle 4, 6 so that this composite 2 covers the corresponding framework 3, 5, 5′. Once the needle 4, 6 was filled with the composite 2, the syringe 7 was retracted and this needle 4, 6 was immersed in an aqueous gelling bath 9 based on polycations, preferably of calcium (16 mM NaCl, 20 mM CaCl₂). As can be seen in FIG. 2, the gelling of the composite 2 was for example carried out by addition dropwise to this gelling bath 9 contained in a flask 10, and at the end of a relatively long time (preferably over at least three days) beads 11 of crosslinked alginate gel were thus obtained with an average diameter of between 2 mm and 3 mm.

“Strips” of composite 2 covering the framework 3, 5, 5′ over a part of its axial length have thus been obtained, this coverage length lying between 1 cm and 5 cm. The photographs of FIGS. 9 and 10 show these coverages; it should be noted that, for better visualization of the composite 2, these photographs were taken with the layer of alginate 2a alone, without the nanoparticles 2b which would have obscured the composite 2, preventing the framework 3, 5′ from being seen clearly.

Thus, the frameworks 5, 5′ having the openings 5b, 5′b are partially embedded in the composite 2, the latter extending on both sides of these frameworks 5, 5′ and through these openings 5b, 5′b. The framework 5 delimits a tubular internal volume filled with composite 2, which extends beyond the said internal volume through the openings 5b.

In order to carry out sampling of targets 1 in the bodily fluid 12, the syringe 7 is again assembled on the needle 6 filled with crosslinked composite 2 covering the framework 3, 5, 5′, in order to insert the needle 6 into the vein 12, as illustrated in FIG. 3 in which the framework 5 and the composite 2 covering it have been represented schematically in the internal position (i.e. not as far as the open end of the needle 6). After the needle 6 has entered the vein 12, the framework 5 covered with the composite 2 is pushed in translation along the needle 6 so that it emerges beyond the end of the latter in the partially external position of FIG. 4, and the nanoparticles 2b embedded in the composite 2 are thus in contact with the targets 1 contained in the vein 12. A liquid, preferably an injectable liquid, may be arranged between the thrust member and the framework 5, 5′ in order to safeguard the composite 2 during the pushing.

As illustrated in the photograph of FIG. 6, it will be noted that the composite 2 (shown as bright regions) covers the frameworks 5, 5′ according to the invention, on the one hand on their faces 5a, 5′a but also on their faces lying on the other side of the framework. These frameworks 5, 5′ have a thickness of between 10 μm and 100 μm.

Comparative tests of washing with water the composites 2 respectively covering the wire framework 3 of FIGS. 7 and 9, the framework 5 according to FIGS. 5 and 6 and the framework 5′ according to FIGS. 8 and 10 were carried out. For washing these three composites 2, a nozzle spraying deionized water (DI water) for an equal period of one minute was used.

The photographs of FIGS. 11 and 12 demonstrate the results obtained for these frameworks 3 and 5′. As can be seen particularly in the lower right portion of FIG. 11, the alginate gel has partially detached from the framework 3 not according to the invention, whereas FIG. 12 conversely shows that the alginate gel of the composite 2 has resisted this washing well and still covers the external face of the framework 5′ with a uniform thickness over the entire axial length of this framework 5′. The situation is the same for the framework 5 of FIGS. 5 and 6.

These comparative tests therefore show that the risks of releasing fragments of polymer from the composite 2 into the bodily liquid are indeed minimized by virtue of the micro-openworked frameworks 5, 5′ according to the invention.

It will be noted that the crosslinked composite 2, which covers the framework 5, 5′ according to the exemplary embodiments of FIGS. 5, 6, 8, 10, 12 over a part of its axial length, was obtained by using the aforementioned preferential mass fraction of 1.5% alginate in the aqueous dispersion. The viscosity of this crosslinked composite 2, consisting of the alginate layer 2a at 1.5% strength, in which the nanoparticles 2b are embedded, was about 250 mPa·s (viscosity measured by a cone and plate rheometer), and its Young's modulus (calculated on the basis of compression tests carried out with a rheometer) was about 77 kPa, making it possible to contribute to good attachment of the composite 2 to the microstructured framework 5, 5′.

1. Sampling device configured to be inserted into a hollow tubular endpiece of a needle or catheter type, and to emerge from the endpiece with a view to contact with a bodily fluid containing biological targets to be sampled, the device comprising:
a framework microstructured by openings, and
a biocompatible and porous crosslinked polymer layer which comprises capture supports adapted to capture the said targets and which is adapted to retain these supports from the fluid and to let through only fluid particles including these targets with a size of less than a cutoff size,
the said polymer layer filling all or some of the said openings, so as to be retained by the said framework,
wherein the said framework is substantially undeformable between positions in which it is inserted into the endpiece and in which it emerges from the latter.

2. Sampling device according to claim 1, wherein the framework is circumscribed by at least one cylindrical surface having a largest transverse dimension of between 500 μm and 2 mm.

3. Sampling device according to claim 1, wherein the framework has an external face and delimits an internal volume, the said polymer layer extending through the said openings from the said internal volume to the said external face and beyond the latter.

4. Sampling device according to claim 1, wherein the said framework has a single longitudinal symmetry axis which is intended to be parallel to that of the said endpiece, the said framework maintaining an overall tubular geometry in the said positions.

5. Sampling device according to claim 1, wherein the said crosslinked polymer layer has a viscosity, measured by a cone and plate rheometer, which is equal to or greater than 100 mPa·s.

6. Sampling device according to claim 1, wherein the said framework is of the latticed, woven or plaited type, comprising a multitude of the said openings, separated in pairs by a pitch of between 30 μm and 60 μm.

7. Sampling device according to claim 3, wherein the framework is circumscribed by at least one cylindrical surface having a largest transverse dimension of between 500 μm and 2 mm, and wherein the said framework has a thickness of between 10 μm and 100 μm, the said at least one cylindrical surface having a substantially elliptical or circular cross section.

8. Sampling device according to claim 4, wherein the framework has an external face and delimits an internal volume, the said polymer layer extending through the said openings from the said internal volume to the said external face and beyond the latter, and wherein the said external face and/or an internal face of the framework furthermore has or have indentations and/or reliefs which are separate from the said openings and which have dimensions of between 20 μm and 90 μm.

9. Sampling device according to claim 4, wherein the framework has an external face and delimits an internal volume, the said polymer layer extending through the said openings from the said internal volume to the said external face and beyond the latter, and wherein the said polymer layer forms, with respect to the said external face of the framework, an external coating substantially coaxial with this framework and having a thickness of between 50 μm and 300 μm.

10. Sampling device according to claim 1, wherein the said crosslinked polymer layer has a Young's modulus, measured on the basis of compression tests carried out with a rheometer, of between 50 kPa and 270 kPa inclusive.

11. Sampling device according to claim 1, wherein the said framework is embedded in the said polymer layer over a part of its axial length lying between 1 mm and 5 cm, this polymer layer having a volume of between 1 ml and 10 ml.

12. Sampling device according to claim 1, characterized in that:
the said framework is made of metallic material silicon or a polymer material such as a silicone,
the said polymer layer is based on at least one biocompatible polymer with reversible gelling, selected from the group consisting of alginate gels, copolymers of alginate and poly-L-lysine, chitosan, agarose, cellulose, poly(trimethylammonium ethylacrylate methyl sulfate)-b-poly(acrylamide), poly(hydroxyethylmethacrylate (HEMA), poly(hydroxyethylmethacrylate-methyl methacrylate (HEMA-MMA) and other copolymers based on methacrylate, polyethylene glycols, copolymers of acrylonitrile and polyethylene glycol, polysaccharides and mixtures thereof, and in that
the framework is provided on its surface with functional groups creating chemical bonds between the framework and the polymer layer, preferably carboxylic acid or amine groups in the case in which the framework is metallic for bonding with hydroxyl groups of this layer.

13. Sampling system comprising:
a hollow tubular endpiece of a needle or catheter type, which has an internal diameter of between 500 μm and 2 mm,
a sampling device inserted into the endpiece and capable of emerging by sliding from an end of this endpiece with a view to contact with a bodily fluid containing biological targets to be sampled, and
a thrust member capable of making the said sampling device slide reversibly out of the said endpiece,
wherein said device is as defined in claim 1, this device optionally being provided with a means for connection to the said end of the endpiece.

14. Sampling system according to claim 13, wherein the said thrust member is of the syringe type, comprising:
a pump body, in which the endpiece is mounted, and
a rod which can be inserted into the said endpiece in order to make the said sampling device slide therein.

15. Method for manufacturing a sampling device according to claim 1, wherein the method comprises the following steps:
a) preparation of an uncrosslinked polymer composite incorporating the said capture supports and the said uncrosslinked polymer layer covering them,
b) insertion of the said framework, without this composite, into a tubular mold, optionally with connection of the framework to a sampling end of the endpiece,
c) assembly of the endpiece containing this framework in a sampling member of the syringe type,
d) take-up of the uncrosslinked composite prepared in a) by this sampling member, in order to inject this composite inside the endpiece in contact with the framework, then
e) crosslinking in a gelling bath of the endpiece which is filled with the uncrosslinked composite injected in d) and which has previously been extracted from this sampling member, in order to obtain the said crosslinked polymer layer fixed to the framework.

16. Manufacturing method according to claim 15, wherein step a) comprises:
a1) dispersion in an aqueous buffer solution of the said capture supports comprising magnetic or non-magnetic functionalized nanoparticles, then
a2) addition under agitation to the dispersion obtained in a1) of at least one biocompatible polymer with reversible gelling, in order to obtain the said uncrosslinked composite in which these nanoparticles are embedded.
